SEQUENCE LISTING (10 sequences)

SEQ ID NO: 1 - DNA, 46 bp, Artificial Sequence
  Description of Artificial Sequence Synthetic oligonucleotide
atctcattctgcgcagctttctcgagaaagctgcgcagaatgagat

SEQ ID NO: 2 - DNA, 48 bp, Artificial Sequence
  Description of Artificial Sequence Synthetic oligonucleotide
tgcgccaacacagaaattattctcgagaataatttctgtgttggcgca

SEQ ID NO: 3 - DNA, 52 bp, Artificial Sequence
  Description of Artificial Sequence Synthetic oligonucleotide
ccggccagtccagatatttcacattctcgagaatgtgaaatatctggactgg

SEQ ID NO: 4 - DNA, 54 bp, Artificial Sequence
  Description of Artificial Sequence Synthetic oligonucleotide
ccgggaagcgctacttggtcaaattctcgagaatttgaccaagtagcgcttctt

SEQ ID NO: 5 - DNA, 20 bp, Artificial Sequence
  Description of Artificial Sequence Synthetic primer
gaggatgaggtggaacgtgt

SEQ ID NO: 6 - DNA, 20 bp, Artificial Sequence
  Description of Artificial Sequence Synthetic primer
agaagtgacgcagccctcta

SEQ ID NO: 7 - DNA, 21 bp, Artificial Sequence
  Description of Artificial Sequence Synthetic primer
aagggattaccagcgaacttg

SEQ ID NO: 8 - DNA, 22 bp, Artificial Sequence
  Description of Artificial Sequence Synthetic primer
cttcagcatgtttgctcagact

SEQ ID NO: 9 - DNA, 20 bp, Artificial Sequence
  Description of Artificial Sequence Synthetic primer
aacgacacaacaagccggaa

SEQ ID NO: 10 - DNA, 21 bp, Artificial Sequence
  Description of Artificial Sequence Synthetic primer
tggagccctcaatcagaacct

(19) United States
(12) Patent Application Publication
(10) Pub. No.: US 2017/0165363 A1
(43) Pub. Date: Jun. 15, 2017

(54) CANCER CELL MIGRATION INHIBITORS AND THEIR USE IN THERAPEUTIC TREATMENTS

(71) Applicant: THE JOHNS HOPKINS UNIVERSITY, Baltimore, MD (US)

(72) Inventors: Denis Gaston Wirtz, Washington, DC (US); Hasini Jayatilaka, Baltimore, MD (US)

(21) Appl. No.: 15/372,796

(22) Filed: Dec. 8, 2016

Related U.S. Application Data
(60) Provisional application No. 62/266,358, filed on Dec. 11, 2015.

(51) Int. Cl. A61K 39/395; G01N 33/574; A61K 31/7088; C12N 15/113; C07K 16/28; A61K 31/18

(52) U.S. Cl. CPC A61K 39/3955; C07K 16/2866; A61K 31/18; A61K 31/7088; C12N 15/1136; G01N 33/574; C07K 2317/24; C07K 2317/76; C12N 2320/31; C12N 2310/531; G01N 2333/5412; G01N 2333/5421

(57) ABSTRACT

Described are methods of treating or preventing cancer in patients by administering Interleukin 6 (IL-6) inhibitor and Interleukin 8 (IL-8) inhibitor, in a concentration ratio range to inhibit the migration of cancer cells.

REFERENCE TO RELATED APPLICATIONS This application claims the benefit of U.S. Provisional Patent Application No. 62/266,358 filed on Dec. 11, 2015, that is hereby incorporated by reference for all purposes as if fully set forth herein.

### STATEMENT OF GOVERNMENTAL INTEREST

This invention was made with government support under grant nos. CA143868 and CA174388, awarded by the National Institutes of Health. The government has certain rights in the invention.

### INCORPORATION-BY-REFERENCE OF MATERIAL SUBMITTED ELECTRONICALLY

The instant application contains a Sequence Listing which has been submitted in ASCII format via EFS-Web and is hereby incorporated by reference in its entirety. Said ASCII copy, created on Nov. 22, 2016, is named P13909_SL.txt and is 2,570 bytes in size.

### BACKGROUND OF THE INVENTION

Despite multimodal cancer therapy, a large number of patients with early-stage cancers experience distant metastases. Metastasis—the spread of cancer cells from the primary organ and colonization at distal sites through the vascular and lymphatic systems—is the least understood process involved in tumorigenesis. Proliferation and migration are two important processes in the metastatic cascade that separately have been studied extensively. These processes are typically thought of as mutually exclusive as metastatic invasive tumor cells seem to delay proliferation during migration. Previous studies have also suggested that proliferative and migratory tumorigenic cells display two distinct transcriptional signatures. However, studies are yet to identify whether cancer cell proliferation and migration could be functionally coupled or exclusive of each other in the tumor microenvironment (TME).

Secreted proteins, such as growth factors and cytokines, in the TME, which includes stromal and immune cells and a collagen-rich extracellular matrix, drive intercellular communications, which mediate physio-pathologically relevant processes, including cancer-induced angiogenesis, de-differentiation, and metastasis. For instance, the highly invasive nature of human metastatic glioblastomas in brain tumors has been attributed to its unique secretomic profile. However, this secretomic profile is likely to change as cancer cells proliferate and tumors progress to a higher grade, suggesting that migration may be dependent on proliferation. Hence, the contribution of proliferation induced crowding, or cell density, may be a significant, yet largely unidentified TME parameter that affects cancer cell migration. Understanding the biological and physiological parameters of cancer cell migration may result in the identification of new protein targets for chemotherapy development that could enhance cancer patient care and win the fight against cancer.

### SUMMARY OF THE INVENTION

The present invention identified two secreted, cellular proteins, Interleukin 6 (IL-6) and Interleukin 8 (IL-8), that must be in a specific concentration ratio range to enhance cancer cell migration. Specific protein inhibitors were used to demonstrate altering this ratio inhibits migration of cancer cells both in vitro and in vivo. Surprisingly, it was discovered that using an IL-6 inhibitor and IL-8 inhibitor in combination inhibited this synergistic cancer cell migration pathway to a much greater extent than adding one of the two agents alone. Other inhibitors of cancer cell migration suitable for the present invention include inhibitors of JAK2, STAT3, WASF3, ARP2/3 complex. An inhibitor of the present invention may inhibitor the gene expression or protein activity of the following genes IL-6, IL-8, JAK2, STAT3, WASF3, ARP2/3 or a combination thereof.

One embodiment of the present invention is a method for inhibiting the migration of cancer cells comprising the following steps: providing a mixture comprising an IL-6 inhibitor and an IL-8 inhibitor; applying the mixture to cancer cells, and inhibiting the migration of the cancer cells. The method is performed in vitro or in vivo, such as administering the mixture to a subject. IL-6 inhibitors used in the methods of the present invention may be a shRNA such as SEQ ID NO:1, SEQ ID NO:3, or a combination thereof, as examples. IL-6 inhibitors may be antibodies such as a recombinant humanized, anti-human IL-6 receptor monoclonal antibody. IL-8 inhibitors suitable for the methods of the present invention may be a shRNA such as SEQ ID NO:2, SEQ ID NO:4, or a combination thereof, as examples. IL-8 inhibitors may be antibodies such as one that is able to inhibit the IL-8 receptor. Alternatively, IL-8 receptors inhibitors may be a chemical such as (2R)-2-[4-(2-methylpropyl)phenyl]-N-methylsulfonylpropanamide, for example. Cancers cells used in the present invention may be any type of cancer cell such as cells of liver metastases or lung metastases. Methods of the present invention wherein the inhibition of migration of cancer cells is observed by comparing cells that have undergone a method of the present invention with reference cancer cells substantially free of the inhibitors of cancer cell migration of the present invention including inhibitors of IL-6, IL-8, JAK2, STAT3, WASF3, ARP2/3, or a combination thereof.

Another embodiment of the present invention is a method of determining cancer patient longevity comprising: providing a biological sample from a cancer patient; placing the biological sample in contact with an IL-6 and IL-8 binding agent; and determining the concentration of IL-6 and 11-8 in the biological sample. The biological sample could be blood, tissue, and/or cells. The IL-6 and IL-8 binding agent may be an antibody or aptamer that specifically bind to IL-6 or IL-8 respectively, as examples. The binding agent may be attached to a detection complex such as a complex able to produce fluorescents as an example. The test will indicate cancer patient longevity is enhanced when the concentration ratio of IL-6/IL-8 in the biological sample is similar to that seen in patients without cancer.

Another embodiment of the present invention is a method of treating or preventing cancer in a subject comprising the following steps: providing a mixture comprising an IL-6 and an IL-8 inhibitor; administering the mixture to a subject with cancer; and treating or preventing cancer.

Another embodiment of the present invention is a method for inhibiting cancer cell migration comprising the following steps: providing a mixture comprising an inhibitor of a gene from the group consisting of JAK2, STAT3, WASF3, ARP2/3 complex or a combination thereof applying the mixture to cancer cells; and inhibiting the migration of the cancer cells.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The term “activity” refers to the ability of a gene to perform its function such as Indoleamine 2,3-dioxygenase (an oxidoreductase) catalyzing the degradation of the essential amino acid tryptophan (trp) to N-formyl-kynurenine.

The term “antibody,” as used in this disclosure, refers to an immunoglobulin or a fragment or a derivative thereof, and encompasses any polypeptide comprising an antigen-binding site, regardless of whether it is produced in vitro or in vivo. The term includes, but is not limited to, polyclonal, monoclonal, monospecific, polyspecific, non-specific, humanized, single-chain, chimeric, synthetic, recombinant, hybrid, mutated, and grafted antibodies. Unless otherwise modified by the term “intact,” as in “intact antibodies,” for the purposes of this disclosure, the term “antibody” also includes antibody fragments such as Fab, F(ab′)2, Fv, scFv, Fd, dAb, and other antibody fragments that retain antigen-binding function, i.e., the ability to bind, for example, PD-L1, specifically. Typically, such fragments would comprise an antigen-binding domain.

The terms “antigen-binding domain,” “antigen-binding fragment,” and “binding fragment” refer to a part of an antibody molecule that comprises amino acids responsible for the specific binding between the antibody and the antigen. In instances, where an antigen is large, the antigen-binding domain may only bind to a part of the antigen. A portion of the antigen molecule that is responsible for specific interactions with the antigen-binding domain is referred to as “epitope” or “antigenic determinant.” An antigen-binding domain typically comprises an antibody light chain variable region (V_{L}) and an antibody heavy chain variable region (V_{H}), however, it does not necessarily have to comprise both. For example, a so-called Fd antibody fragment consists only of a V_{H} domain, but still retains some antigen-binding function of the intact antibody.

Binding fragments of an antibody are produced by recombinant DNA techniques, or by enzymatic or chemical cleavage of intact antibodies. Binding fragments include Fab, Fab′, F(ab′)2, Fv, and single-chain antibodies. An antibody other than a “bispecific” or “bifunctional” antibody is understood to have each of its binding sites identical. Digestion of antibodies with the enzyme, papain, results in two identical antigen-binding fragments, known also as “Fab” fragments, and a “Fc” fragment, having no antigen-binding activity but having the ability to crystallize. Digestion of antibodies with the enzyme, pepsin, results in a F(ab′)2 fragment in which the two arms of the antibody molecule remain linked and comprise two-antigen binding sites. The F(ab′)2 fragment has the ability to crosslink antigen. “Fv” when used herein refers to the minimum fragment of an antibody that retains both antigen-recognition and antigen-binding sites. “Fab” when used herein refers to a fragment of an antibody that comprises the constant domain of the light chain and the CHI domain of the heavy chain.

By “ameliorate” is meant decrease, suppress, attenuate, diminish, arrest, or stabilize the development or progression of a disease.

By “alteration” is meant a change (increase or decrease) in the expression levels or activity of a gene or polypeptide, such as IL-6, IL-8, JAK2, STAT3, or WASF3, as detected by standard art known methods such as those described herein. As used herein, an alteration includes a 10% change in expression levels, preferably a 25% change, more preferably a 40% change, and most preferably a 50% or greater change in expression levels. “

By “analog” is meant a molecule that is not identical, but has analogous functional or structural features, for example such as an analog to a chemical compound such as (2R)-2-[4-(2-methylpropyl)phenyl]-N-methylsulfonylpropanamide or Reparixin. Another example is a polypeptide analog that retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By anti-ARP2/3 complex” is meant an antibody that selectively binds to ARP2/3 complex.

By “anti-IL-6 antibody” is meant an antibody that selectively binds Interleukin 6 or a part thereof.

By “anti-IL-8 antibody” is meant an antibody that selectively binds Interleukin 8 or a part thereof.

By “anti-JAK2” is meant an antibody that selectively binds to JAK2.

By “anti-STAT3” is meant an antibody that selectively binds to STAT3.

By “anti-WASF3” is meant an antibody that selectively binds to WASF3.

By “ARP2/3 complex” is meant a multi subunit protein complex that plays a major role in the regulation of the actin cytoskeleton.

By “IL-6” is meant an Interleukin-6 gene and its gene products including mRNA, and the interleukin 6 (IL-6) protein. An example is the human IL-having a Gene ID: 3569 on a (National Center for Biotechnology Information) NCBI database.

By “IL-8” is meant an Interleukin-8 gene and its gene products including mRNA, and the Interleukin-8 (IL-8) protein. An example is the human IL-8 having a Gene ID: 3576 on a (National Center for Biotechnology Information) NCBI database.

By “JAK2” is meant a Janus Kinase 2 gene and gene products including mRNA, and the Janus Kinase 2 (JAK2) protein. An example is the human JAK2 having a Gene ID: 3717 on a (National Center for Biotechnology Information) NCBI database.

By “STAT3” is meant a Signal Transducer and Activator of Transcription 3 (STAT3) gene and its gene products including mRNA, and the Singal Transducer and Activator of Transcription (STAT3) protein. An example is the human STAT3 having a Gene ID: 6774 on a NCBI database.

By “WASF3” is meant a Wiskott-Aldrich Syndrome Protein Family Member 3 (WASF3) gene and its gene products including mRNA, and the WASF3 protein. An example is the human WASF3 having a Gene ID: 10810 on a NCBI database.

By “disease” is meant any condition or disorder that damages or interferes with the normal function of a cell, tissue, or organ. Examples of diseases include cancer.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “express” is meant the ability of a gene to express the gene product including for example its corresponding mRNA or protein sequence (s).

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “Immunoassay” is meant an assay that uses an antibody to specifically bind an antigen (e.g., IL-6, IL-8, ARP2/3 complex, STAT3, JAK2, or WASF3). The immunoassay is characterized by the use of specific binding properties of a particular antibody to isolate, target, and/or quantify the antigen.

The term, “obtaining” as in “obtaining an agent” includes synthesizing, purchasing, or otherwise acquiring the agent.

The term “mAb” refers to monoclonal antibody. Antibodies of the invention comprise without limitation whole native antibodies, bispecific antibodies; chimeric antibodies; Fab, Fab′, single chain V region fragments (scFv), fusion polypeptides, and unconventional antibodies.

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

By “reduces” or “decreases” or “inhibits” is meant a negative alteration of at least 10%, 25%, 50%, 75%, or 100%.

A “reference” refers to a standard or control conditions such as a sample (human cells) or a subject that is a free, or substantially free, of an agent such as one or more inhibitors of the present invention.

By “specifically binds” is meant a compound or antibody that recognizes and binds a polypeptide of the invention, but which does not substantially recognize and bind other molecules in a sample, for example, a biological sample, which naturally includes a polypeptide of the invention.

By “subject” is intended to refer to any individual or patient to which the method described herein is performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

Such treatment (surgery and/or chemotherapy) will be suitably administered to subjects, particularly humans, suffering from, having, susceptible to, or at risk for cancer or disease, disorder, or symptom thereof. Determination of those subjects “at risk” can be made by any objective or subjective determination by a diagnostic test or opinion of a subject or health care provider (e.g., genetic test, enzyme or protein marker, a marker (as defined herein), family history, and the like).

### BRIEF DESCRIPTION OF THE DRAWINGS

FIG. 1A-1K illustrates the effect of cell density on cancer cell motility. A. Phase contrast micrographs demonstrate confluence of human fibrosarcoma cells (HT1080WT) days after initial seeding. B. Cell speed measured at a time lag of 2 min days after initial seeding. C. Average distance to nearest cell (dR) relates density at different days to initial seeding density. D. Randomly selected trajectories of human fibrosarcoma cells (HT1080WT) under different seeding densities of 50, 10, 120 cells/mm³ embedded in a 3D collagen matrix. Phase contrast micrographs demonstrate the confluence at each density. E. Cell velocity measured at a time lag of 2 min at different seeding densities. F. Topology of protrusions for cells embedded in 3D collagen matrices: Oth generation protrusions (N₀) originate from the cell body, 1^{st} generation protrusions (N₁) stem from No and 2^{nd} generation protrusions (N2) stem from N₁. G. Relationship between protrusion frequency and cell density: Protrusion frequency increases with cell density and plateaus at a threshold density of 50 cells/mm³. H. Cell velocity and protrusion frequency are highly correlated. I. Randomly selected trajectories of human carcinoma breast cancer cells (MDA-MB 231) under seeding densities of 10, 50, 120 cells/mm³. J. Cell velocity evaluated at a time lag of 2 min, at five different seeding densities. Cells at high seeding densities (β>50) show a significantly higher velocity than cells seeded at low seeding density (ρ=10). K. Average doubling time at increasing cell density demonstrates that proliferation is independent of cell density.

FIG. 2A-2E illustrates physical cues of cell migration. A. Representative reflection confocal micrograph (RCM) of a 3D collagen matrix in which a HT1080 cell was embedded. B and C. Fiber alignment as measured by image analysis of RCMs and cell density are weakly correlated (B); cell velocity and fiber alignment are also weakly correlated (C). D and E. Interfiber spacing of the collagen matrix and cell density are poorly correlated and so is the cell velocity and average interfiber spacing.

FIG. 3A-3H illustrates biochemical cues of cell cells migration. A. Method to prepare condition medium: medium is incubated for 24 h with a collagen matrix containing a high density of cells, 50 cells/mm³ (HD), which is then filtered using a 0.45 μm filter, and added to a matrix containing a low density of cells, 10 cells/mm³ (LD). B. The addition of conditioned medium (CM) from a matrix containing a high cell density (HD) increases the velocity of cells in a matrix containing a low cell density (LD). The HD cell velocity in the presence of fresh medium (FM) is recapitulated in LD when using CM. C. Secretomic analysis of CM harvested from human fibrosarcoma cells indicates that levels of interleukin 6 (IL-6) and interleukin 8 (IL-8) increase as a function of HT1080 cell density in the matrix, while levels of other major cytokines do not significantly change. D. Secretomic analysis of conditioned medium from human breast carcinoma cells (MDA-MB 231) confirms our observations with HT1080 cells. E and F. Increasing human fibrosarcoma cell density in the matrix increases the concentrations of secreted IL-6 (A) and IL-8 (B), as analyzed by ELISA. G and H. Increasing human carcinoma cell density in the matrix increases the concentrations of secreted IL-6 (A) and IL-8 (B), as analyzed by ELISA.

FIG. 4A-4K illustrates functional influence of IL-6 and IL-8 in cancer cell migration. A and B. The addition of recombinant IL-6 alone (C) or recombinant IL-8 alone (D) do not increase cell velocity. C. The addition of recombinant IL-6 and IL-8 in combination at the precise concentrations found in a matrix containing a high density of 50 cells/mm³ (RM) recapitulates the high velocity observed of human fibrosarcoma cells at high densities. D. Decreased velocity at LD (ρ=10) where cells are exposed to conditioned medium from IL-6 and IL-8 knockdown cells and conditioned medium from a matrix containing a high cell density (HD) with exposure to specific IL-6 and IL-8 functional antibodies compared to control cells exposed to conditioned medium from wild type cells at HD (ρ=50). E. Decreased velocity of the IL-6 and IL-8 knockdown cells at LD (ρ=10) and HD (ρ=50). F. The addition of recombinant IL-6 and IL-8 in combination at the precise concentrations found in a matrix containing a high density of 100 cells/mm^{3 recapitulates} the high velocity observed of human carcinoma cells at high densities. G. Cartoon depicts that IL-6 and IL-8 are required in combination to influence cancer cell motility. H. Decreased velocity of the IL-6R and IL-8R knockdown cells at LD (ρ=10) and HD (ρ=50). I. Cartoon depicts that Tocilizumab and Reparixin can be used to block the cognate receptors of IL-6 and IL-8. J. Individually, Tocilizumab and Reparixin decreased cell velocity of human fibrosarcoma cells embedded in a 3D matrix_at LD (ρ=10) and HD (ρ=50) compared to cells exposed to fresh medium (0). K. Tocilizumab and Reparixin in combination greatly decrease cell velocity of cells embedded in a 3D matrix_at LD (ρ=10) and HD (ρ=50) compared to cells exposed to fresh medium (0).

FIG. 5A-5E illustrates a proposed mechanism of a cancer cell migration process. A. Activity of STAT3 in 3D conditions at LD (ρ=10) and HD (ρ=50). B. Decreased cell velocity of human fibrosarcoma cells embedded in a 3D matrix exposed to JAK2 inhibitor, AG-490, STAT3 inhibitor, S31-201, and ARP 2/3 complex inhibitor, CK 666, at LD (ρ=10) and HD (ρ=50) compared to cells exposed to fresh medium (0). C. Increased expression of WASF3 at HD and cells exposed to recombinant IL-6 and IL-8 in combination at the precise concentrations found in a matrix containing a high density of 50 cells/mm³ D. Cartoon depiction of the IL-6 and the IL-8 signaling pathway to cell motility. E. Clinical data of breast cancer patients representing percent survival for high and low expression of WASF3 and ARP2/3.

FIG. 6A-6H illustrates the effect of cell density on cancer cell motility.

FIG. 7A-7B illustrates mRNA expression.

FIG. 8A-8G illustrates cell speed and IL-6 and IL-8 concentrations.

FIG. 9A illustrates ARP 2/3 expression.

### DETAILED DESCRIPTION OF THE INVENTION

The present invention was first identified through a number of scientific discoveries beginning with a finding that cell migration increases as cells proliferate and cell density increases in a 3D matrix. This increase in cell density caused an increase in the secretion of specific soluble proteins. Using a high-throughput multiplexing cell secretomic profiling assay, the level of 24 different secreted molecules were examined and it was discovered that the secretion of Interleukin 6 (IL-6) and Interleukin 8 (IL-8) specifically increased with cell density in the matrix. IL-6 and IL-8 were determined to be necessary and sufficient to increase cell migration in a cell density dependent manner with no feedback on cell proliferation, an effect specific to cancer cells. Enhanced migration occurred through increased expression of Wiskott-Aldrich syndrome protein family member 3 (WASF3), which in turn regulated actin cytoskeleton dynamics through the recruitment of the ARP2/3 complex, which in turn increased the formation of dendritic cell protrusions, driving enhanced 3D cell migration and connecting cell proliferation to cell migration through a novel, synergistic paracrine signaling pathway.

### Cell Density Enhances Cell Motility

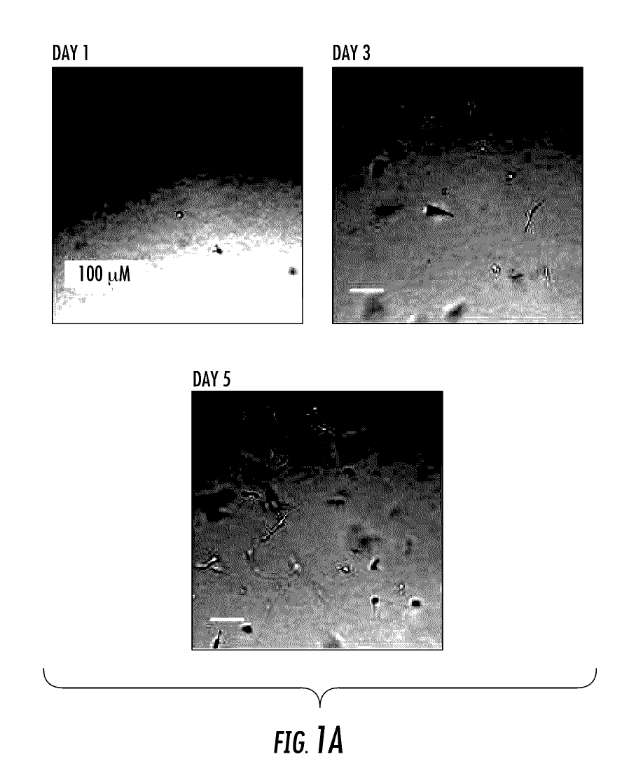

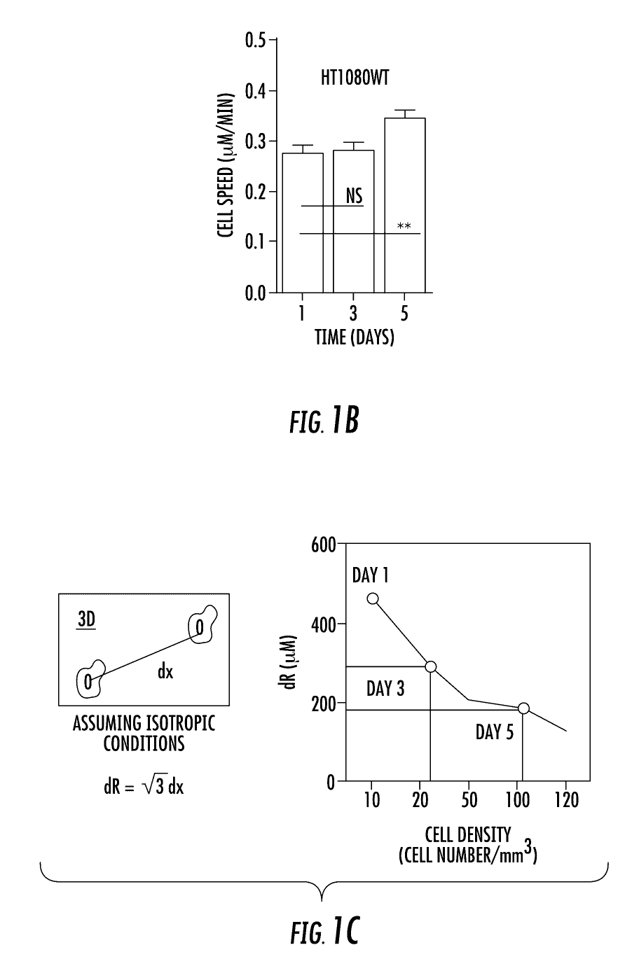

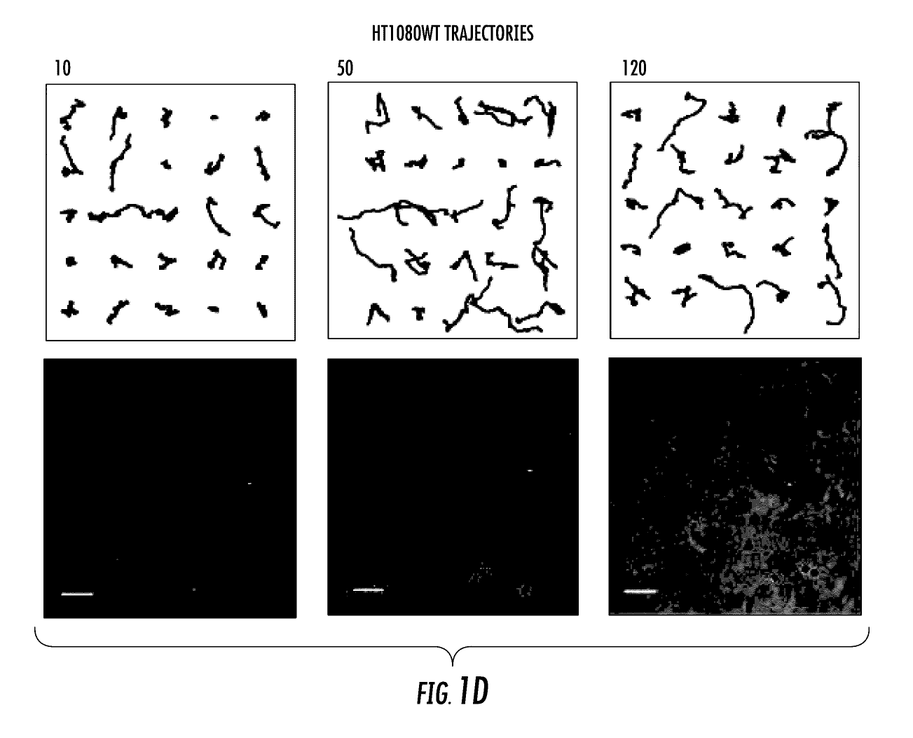

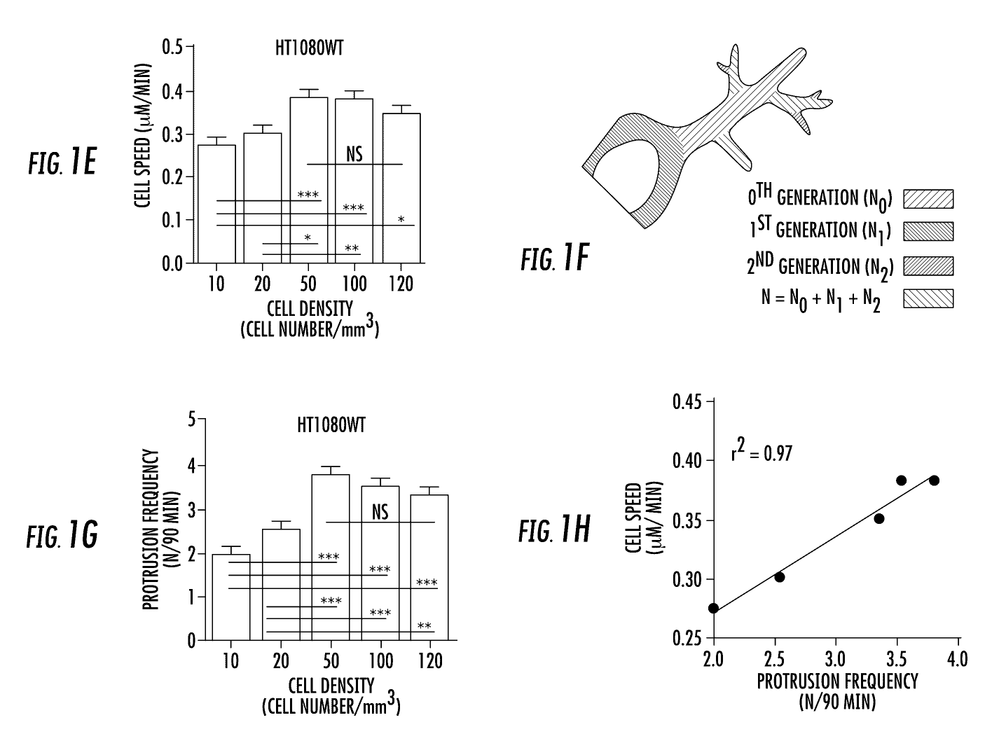

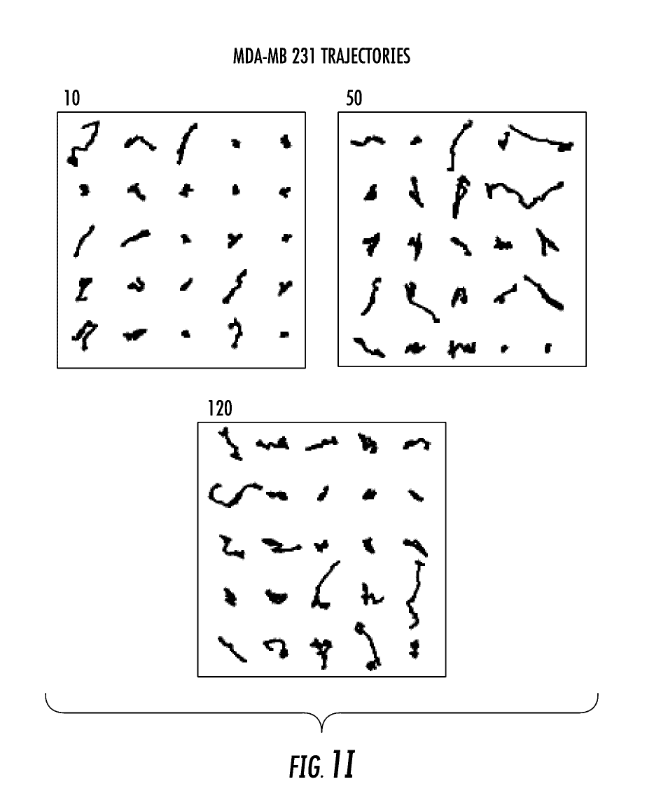

Prior to this invention it was unclear whether cancer cell proliferation and cell migration could be functionally coupled in the tumor microenvironment. The present invention was discovered by assessing the potential role of cell proliferation on cancer cell migration in vitro, using human fibrosarcoma HT1080 cells that were embedded in three dimensional (3D) type I collagen matrices. Collagen I is not only the main extracellular matrix component of connective tissues, but is also enriched in the vicinity of carcinoma and sarcoma tumors. Cell migratory patterns were monitored for 16.5 h using live-cell phase-contrast microscopy at a rate of a 30 frames/h for 5 days. This analysis revealed that fibrosarcoma cells became progressively more motile as cells proliferated and increased local cell density (FIGS. 1, A-C).

To further investigate the role of increased cell density on cancer cell migration, it was determined how increasing cell density in matrices would modulate cell migration. The initial cell densities used in the experiments, ranging from 10 cells/mm³ to 120 cells/mm³, corresponded to average cell-to-cell distances from 470 to 130 μm in the 3D matrix, which were all significantly larger than the average cell size (10-20 μm in diameter). This analysis also revealed that cells became progressively more motile as cell density increased. Cell speed eventually plateaued at a high cell density of 100 cells/mm³ (FIGS. 1, D and E).

Cell motility in 3D matrices is predicted by the ability of cells to form dendritic protrusions. Consistent with these observations, the results suggest that the total number of main and dendritic protrusions generated per unit time by the cells steadily increased and then plateaued with cell density. The cell-density-dependent number of protrusions generated by the cells in 3D matrix strongly correlated with the cell-density-dependent cell velocity (FIGS. 1, F-H).

This remarkable relationship between cell density and cell migration was also found in human metastatic carcinoma cells (MDA-MB 231) and human metastatic glioblastoma cells (U-87). Similar to fibrosarcoma cells, the migration of these two tumorigenic, metastatic cell lines increased with cell density (FIGS. 1, I and J and FIG. S1, A). In contrast, cell-density-dependent migration was not observed in tumorigenic, non-metastatic carcinoma cells (MCF7) and non-tumorigenic cell lines, WI-38 human lung fibroblasts and MCF10A human epithelial cells. (FIGS. S1, B-D).

Importantly, this enhanced migration mediated by cell density in cancer cells did not occur when cells were placed on two-dimensional (2D) collagen-coated substrates (FIGS. S1, E-G). Moreover, in contrast to 3D cell migration, the proliferation of cells in 3D matrices was unaffected by cell density (FIG. 1K and S1H), i.e. cells continued to proliferated at a constant rate regardless of cell density. These results suggest that cell density enhances cell migration, but not proliferation, and that cell-density-dependent migration is unique to tumorigenic cells in 3D microenvironments.

### Enhanced Cell Migration is not Mediated by Properties of the Matrix

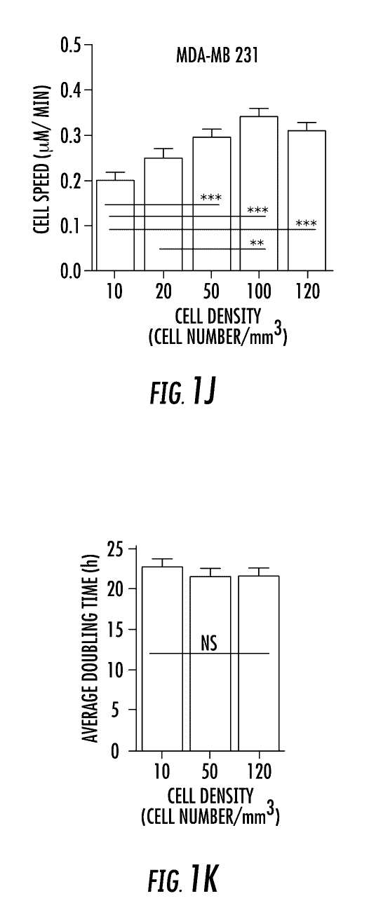

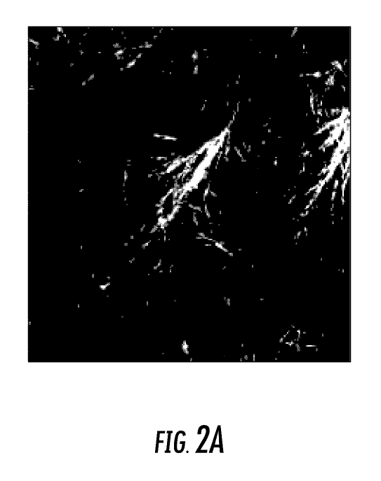

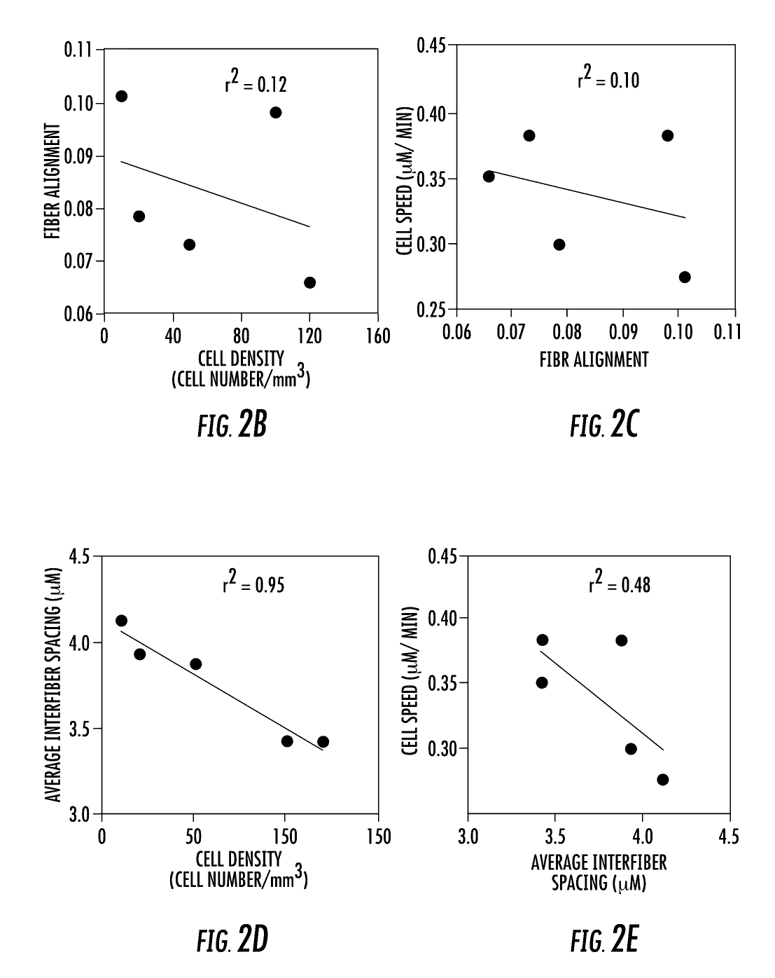

Increased cell density in a 3D matrix may change the physical properties of that matrix, which could promote cell migration. Using reflection confocal microscopy, it was determined if cell density modulated microstructural properties of the 3D collagen I matrix such as interfiber spacing and local fiber alignment. The data demonstrated that local fiber alignment showed poor correlations with cell density and cell speed. Average interfiber spacing showed a poor correlation with cell speed as well. Interestingly, a strong negative correlation was identified between average interfiber spacing and cell density (FIGS. 2 A-E). However, this result was to be expected as cell density increases the forces exerted on the collagen fibrils due to cell movement increases causing the space between the fibrils to decrease. Because of this, we would have expected cell speed to decrease as cell density increased. However, since the cells move faster as cell density increases, this physical property of the collagen matrix cannot modulate cell-density-dependent migration. In sum, this study implicated that the observed enhanced cell migration for increasing cell density cannot be attributed to the changes in the physical properties of the matrix.

### Secretomic Profiles of Matrix-Embedded Cells and Recapitulation

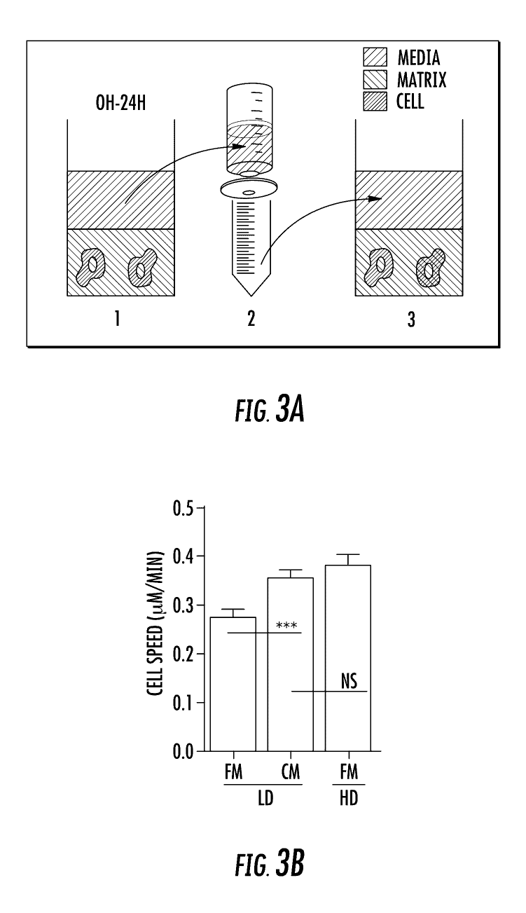

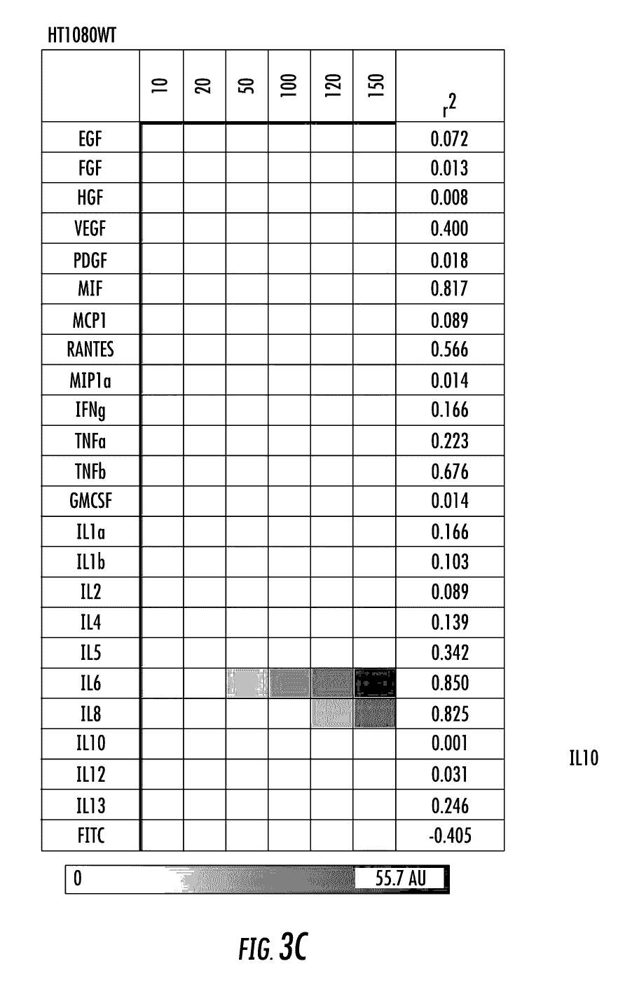

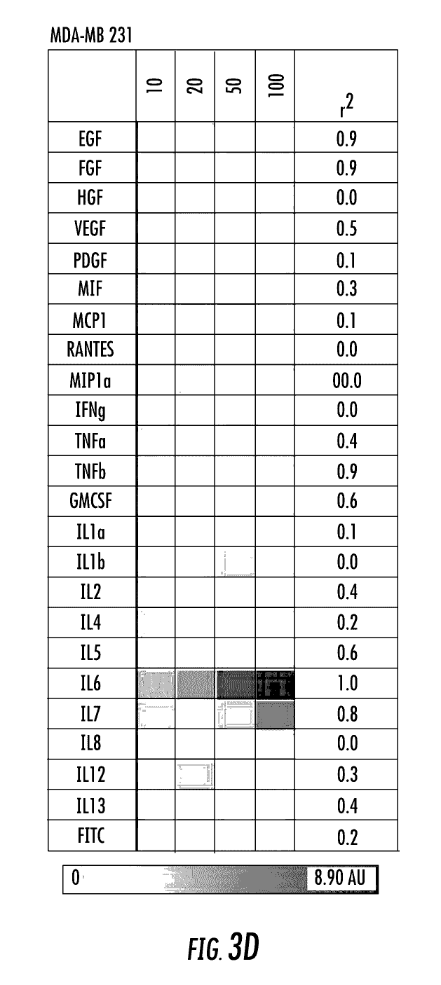

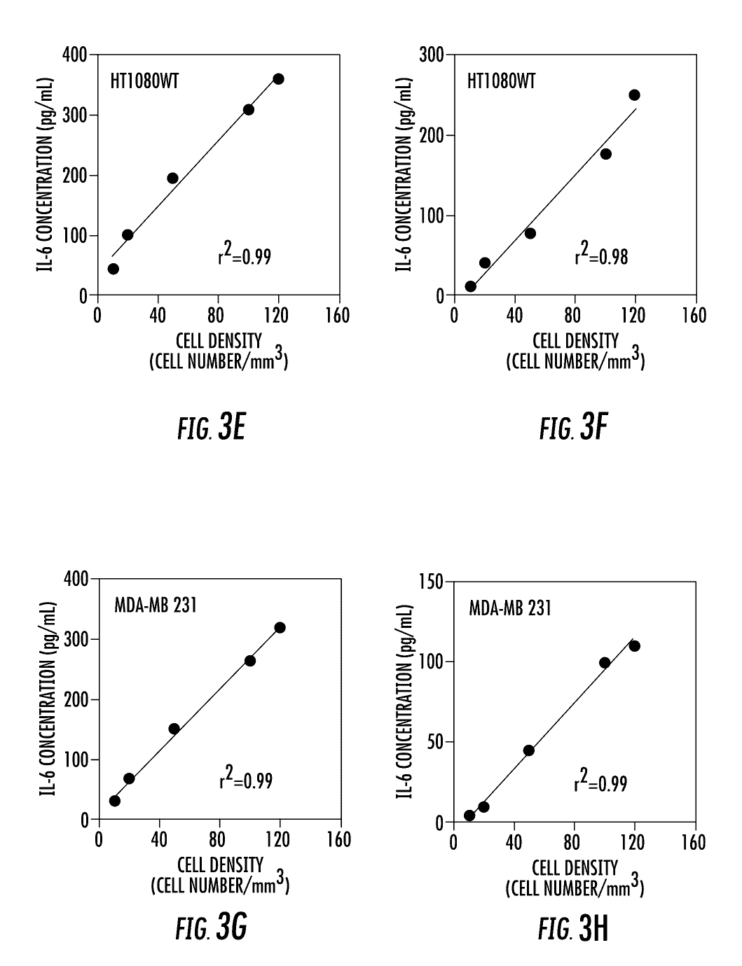

Based on the above results, it was hypothesized that cell-density-dependent migration was regulated by soluble molecules secreted by the cells in a cell-density-dependent manner. To test this hypothesis, conditioned medium collected from a matrix containing a high density of HT1080 cells (50 cells/mm³) was introduced into a matrix containing a low density of HT1080 cells (10 cells/mm³). Enhanced cell velocity was observed at high cell density that could be completely recapitulated at a low cell density by adding condition medium collected from high cell density matrices (FIGS. 3, A and B). This result indicated that soluble molecules secreted by cancer cells in the matrix were sufficient to promote enhanced cell migration.

To identify the soluble factor(s) driving enhanced motility, the secretomic profiles of HT1080 and MDA-MB 231 cells embedded at low and high densities in 3D matrices, using a multiplex antibody microarray assay were measured and analyzed. This assay can simultaneously measure the concentration of 24 soluble molecules secreted by individual cancer cells. The cytokines Interleukin 6 (IL-6) and Interleukin 8 (IL-8) were both expressed in relatively high concentrations and increased with cell density for both HT1080 and MDA-MB 231 cells. Remarkably, all other secreted proteins that were assayed including vascular endothelial growth factor (VEGF), which is a key mediator of angiogenesis in cancer, and hepatocyte growth factor (HGF), which is known to contribute to oncogenesis, tumor progression and tumor metastasis in several cancers, were not increased with increased cell density (FIGS. 3, C and D). Using ELISA, we confirmed our results and determined the precise concentration of the IL-6 and IL-8 at specific cell densities of matrix-embedded HT1080 and MDA-MB 231 cells (FIGS. 3, E-H). Together, this result suggests that IL-6 and IL-8 drive density-dependent cell migration in 3D matrices.

### IL-6 and IL-8 Together are Necessary and Sufficient to Drive Enhanced Cell Migration

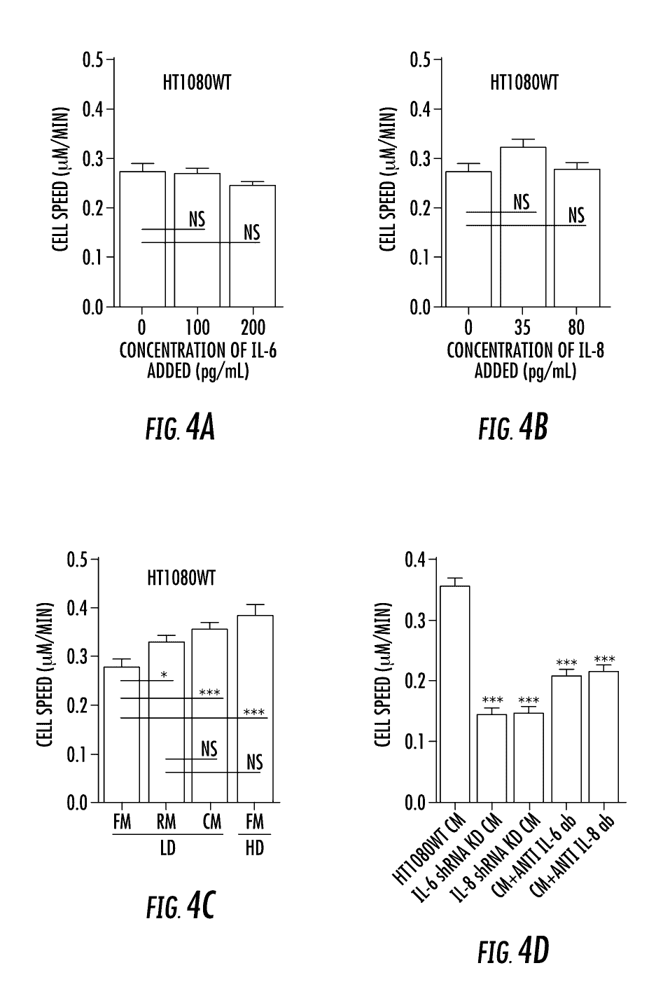

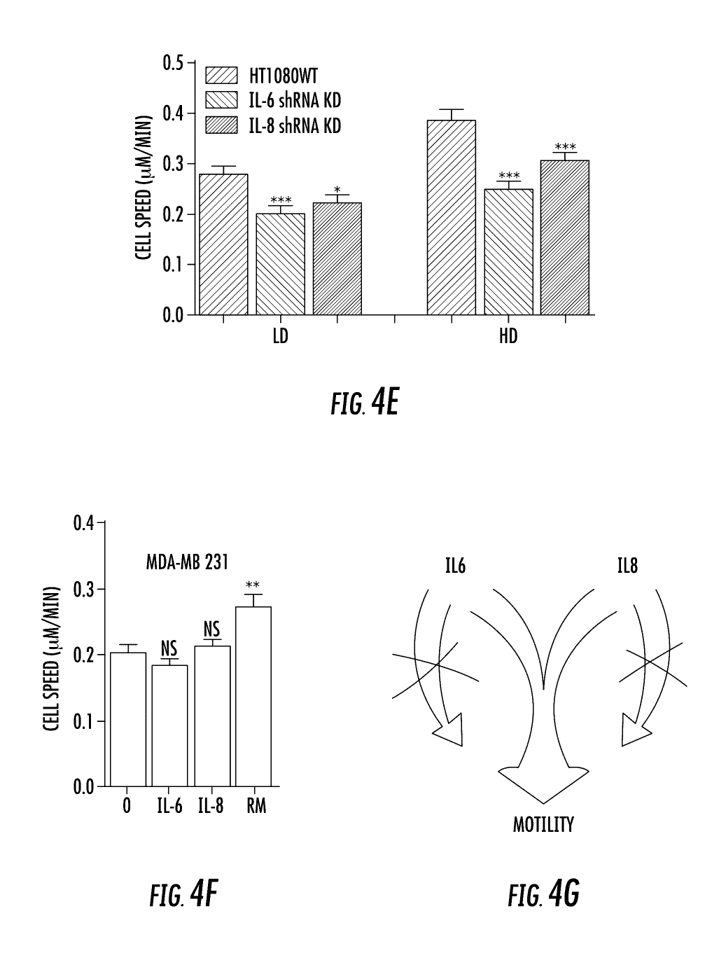

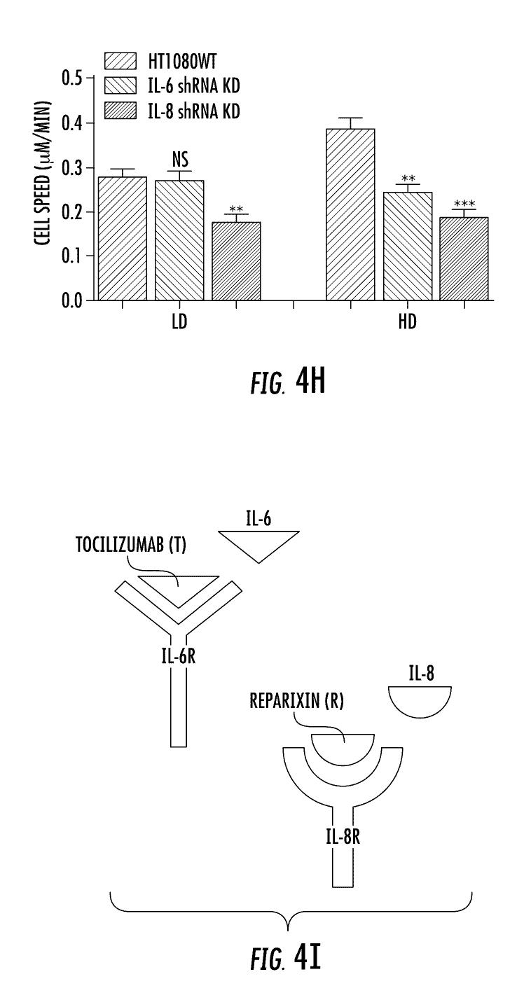

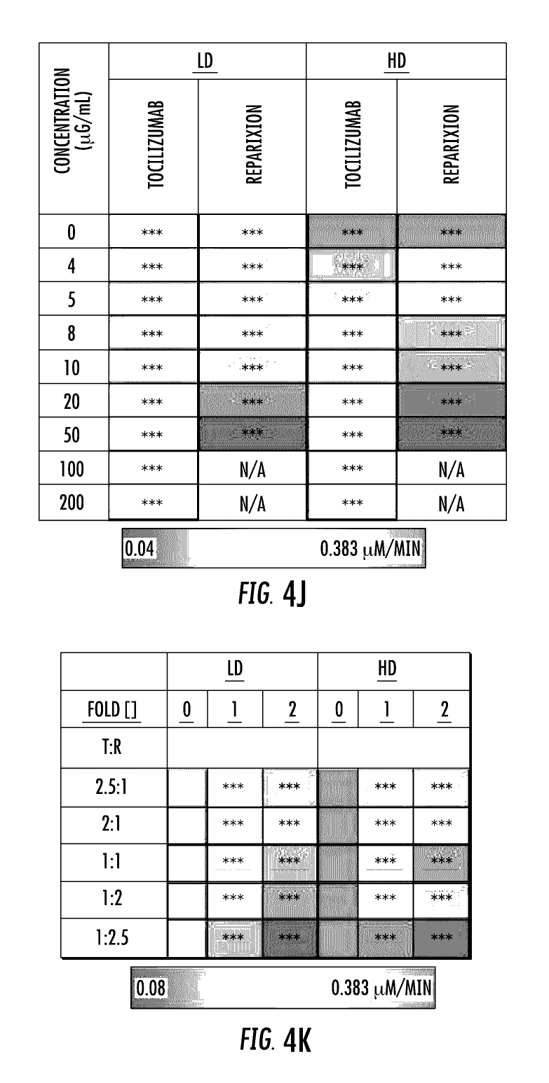

The requirements of IL-6 and IL-8 for cell-density enhanced migration were determined by conducting gain-of-function and loss-of-function experiments. HT1080 cells were seeded at a low density in the matrix to controlled concentrations of human recombinant IL-6 and IL-8. Remarkably, IL-6 or IL-8 alone had no effect on cell migration, even at high concentrations (FIGS. 4, A and B and S3, A and B). In contrast, IL-6 and IL-8, when combined in the precise concentrations found at a high density of 50 cells/mm³ in the stoichiometric ratio of 5:2, induced cells at low density to move the high velocity observed at high cell density. This is consistent with cell velocity observed when exposing cells at low density to condition medium obtained at high density (FIG. 4C). Strikingly, it was also observed that other stoichiometric ratios of IL-6 and IL-8 did not induce the enhanced migration (FIG. S3C). These results indicate that a mixture of IL-6 and IL-8 is sufficient to recapitulate the enhanced migration of cells embedded at high densities in matrices.

To verify that both IL-6 and IL-8 were required to enhance cell-density-dependent migration, we conducted experiments with condition medium from HT1080 cells depleted of IL-6 or IL-8. Depleting either IL-6 or IL-8 prevented the conditioned medium from high density matrices to enhance cell migration of low density matrices (FIG. 4D). Similar results were obtained when we utilized specific IL-6 and IL-8 functional antibodies to block IL-6 and IL-8 secretion into conditioned media. The loss-of-function assays conducted with matrix-embedded cells at low and high cell densities exposed to specific IL-6 and IL-8 functional antibodies and with HT1080 cells depleted of IL-6 and IL-8 through shRNA (over 70% depletion) and embedded in 3D matrices demonstrated that the cell-density-dependent migration patterns observed previously were no longer detected (FIG. 4 E and FIGS. S3E, F and G). These results were confirmed in MDA-MB-231 cells embedded in 3D matrices (FIG. 4F). Interestingly, as with fibrosarcomas, the enhanced cell migration of MDA-MB 231 cells was observed when IL-6 and IL-8 were present in the stoichiometric ratio of 5:2 (FIG. S3D).

Together these results suggest that IL-6 and IL-8 are both required and sufficient in the unique stoichiometric ratio of 5:2 to induce enhanced cancer cell migration caused by proliferation (FIG. 4H).

### Molecular Mechanism of Transduction

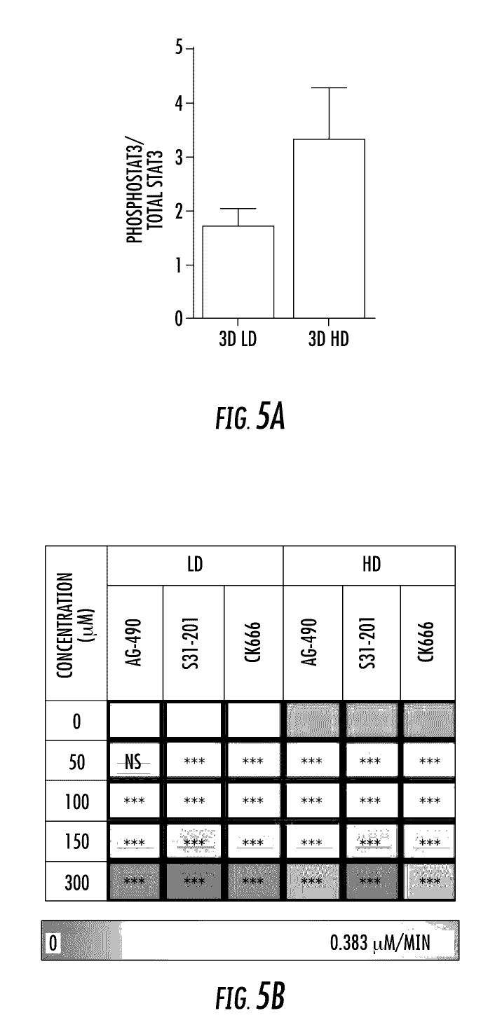

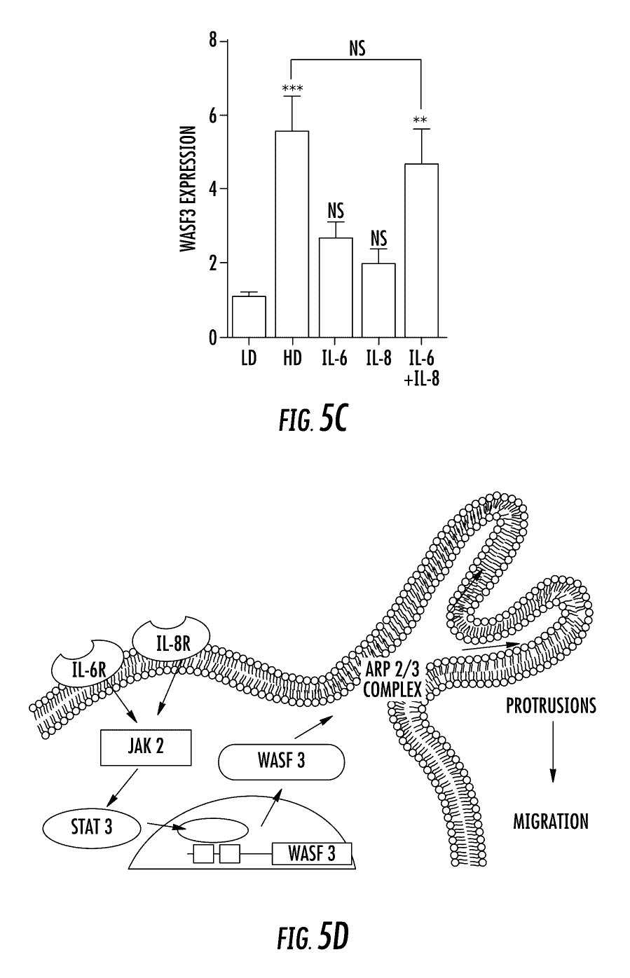

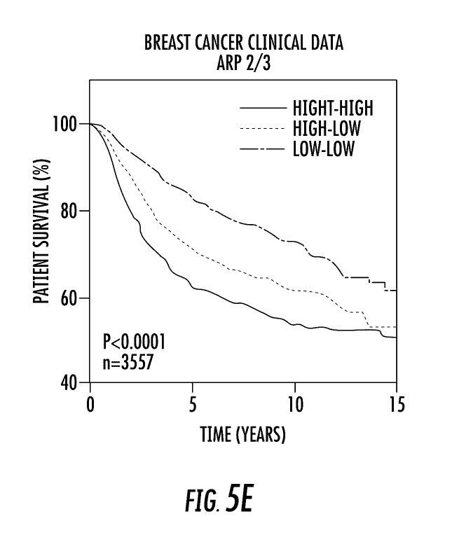

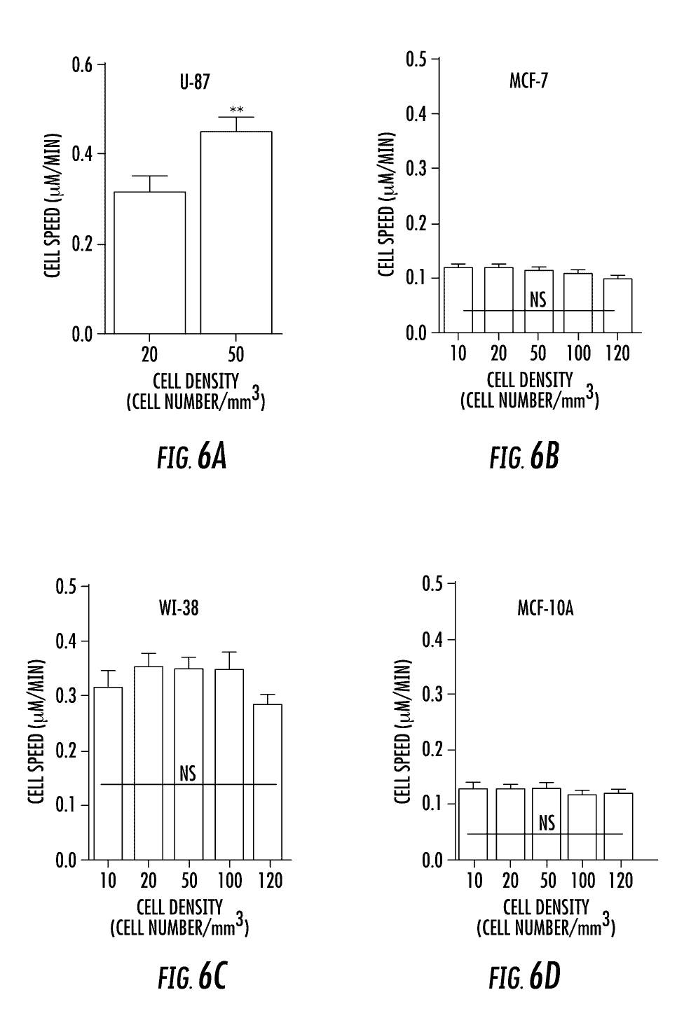

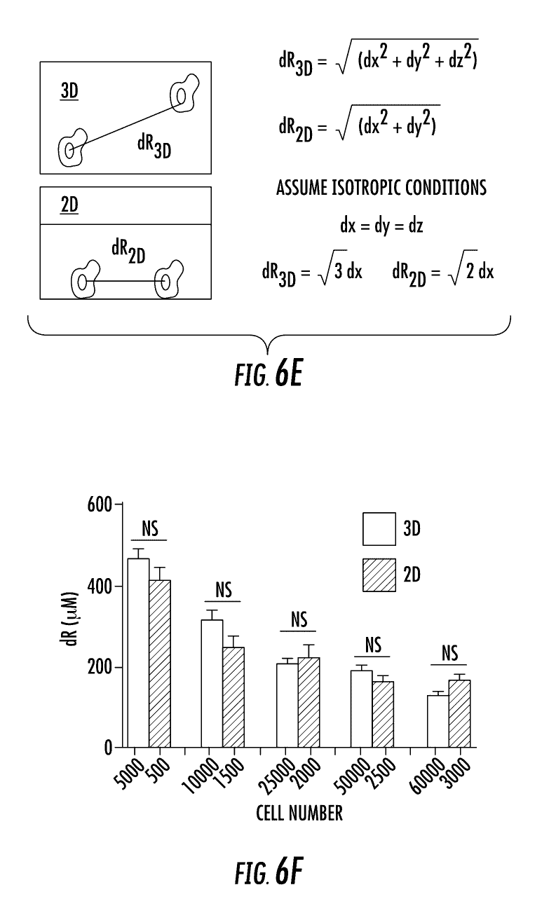

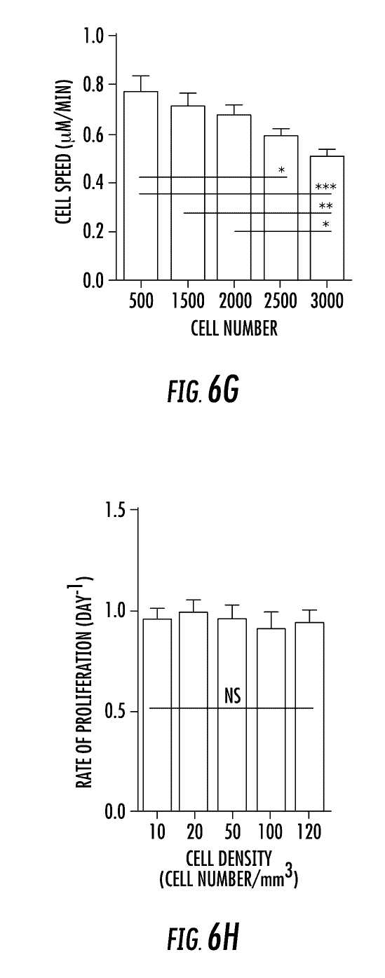

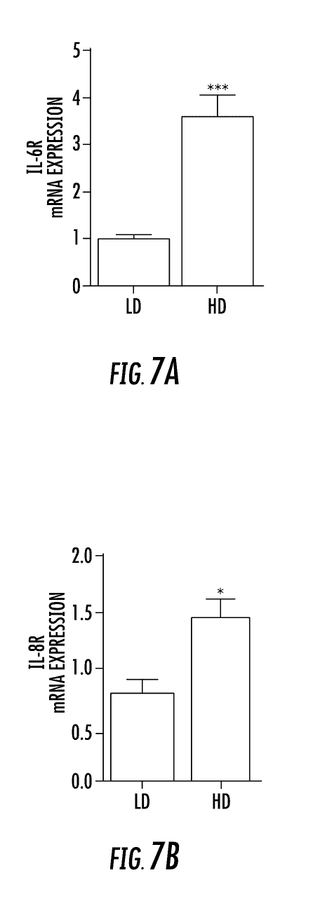

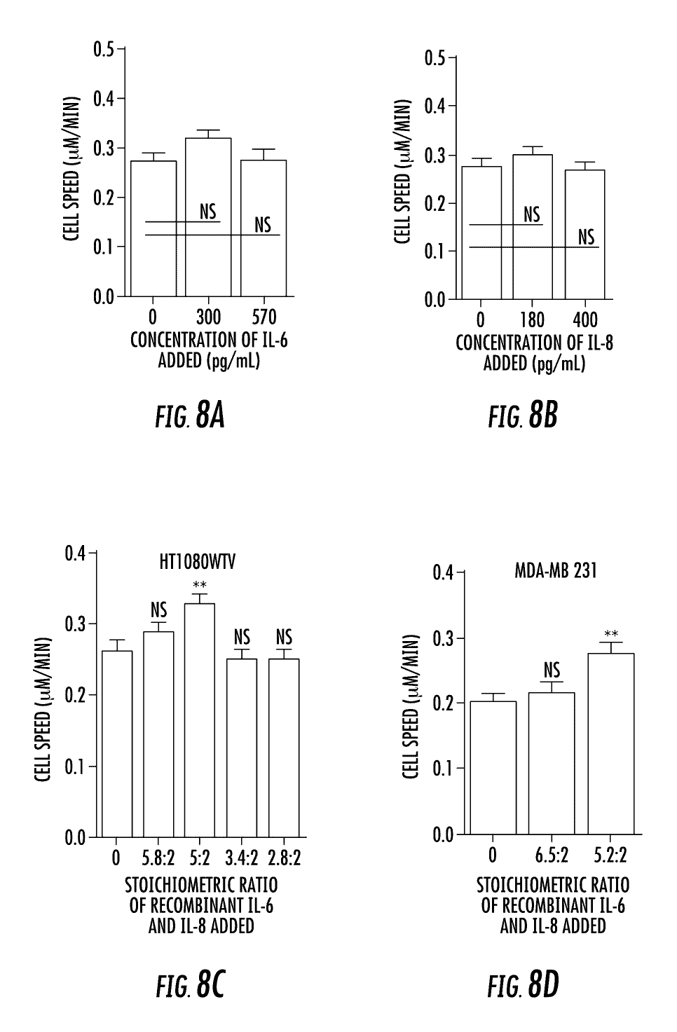

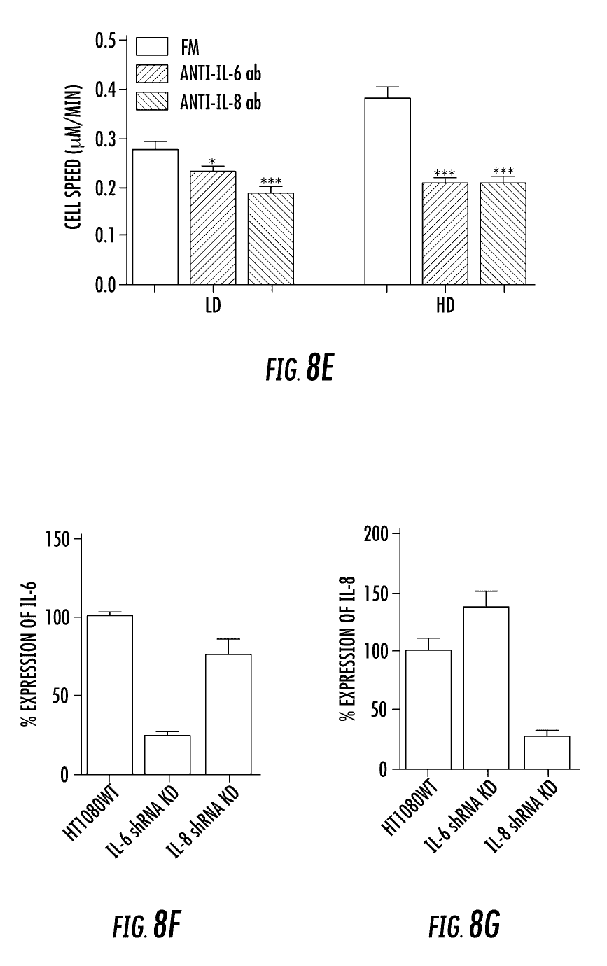

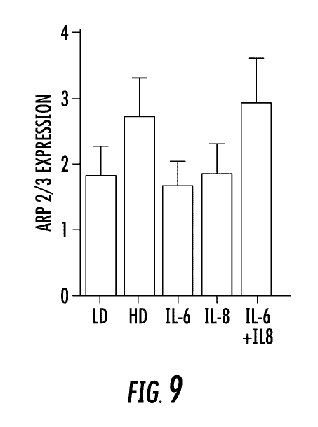

Signal transducer and activator of transcription 3, STAT3, is a transcription factor that is a common downstream effector in the individual pathways of IL-6 and IL-8. Therefore it was hypothesized that STAT3 could regulate cell-density-dependent migration. The activity of STAT3 in HT1080 cells seeded in matrices at a high density was determined to be twice the activity of these cells when seeded at a low density (FIG. 5A).

The ARP2/3 complex nucleates F-actin assembly and mediates dendritic protrusions required for 3D cancer cell migration. Since ARP2/3 is a downstream effector of the STAT3 pathway, it was reasoned that cell-density-dependent migration may be regulated by the ARP2/3 complex through the Janus kinase JAK/STAT3 pathway. Thus, the migration of cells at low and high cell densities exposed to specific JAK2 inhibitor AG-490, STAT3inhibitor S3I-201, and ARP 2/3 complex inhibitor CK666 was examined. Through these inhibitor studies, it was determined that JAK2, STAT3, and the ARP2/3 complex were required for cell-density-dependent migration. Treatment with either of the three inhibitors prevented cell-density-dependent migration (FIG. 5D and S4 A).

It was predicted that Wiskott-Aldrich syndrome protein family member 3 (WASF3) was an important intermediate between STAT3 and the ARP2/3 complex, as it is known to be involved in the regulation of actin cytoskeleton dynamics through the recruitment of ARP2/3 complex. The expression of WASF3 using PCR methods were examined and it was determine that the mRNA level of WASF3 was increased in cells cultured in a high cell density compared to its expression in cells at low cell densities. Also observed was that WASF3 expression of cells at low density exposed to the precise concentration found at a high density of 50 cells/mm³ were comparable to those observed at a high cell density (FIG. 5C). These results suggest that WASF3 is an integral component of the pathway that regulates enhanced migration of cells induced by cell density through the ARP2/3 complex (FIG. 5D and S1 F).

The role of the ARP 2/3 complex on potential clinical contributions for breast cancer and ovarian cancer patients was assessed by utilizing clinical annotations associated with genomic expression data from the Kaplan Meier plotter website. Using their data portal (http://kmplot.com/analysis/), our analysis indicates that patients (n=3557) who expressed higher levels of the ARP2/3 complex had a lower survival rate than those who expressed lower levels of the complex (FIG. 5E and S3 B).

The results of this study suggest that cancer cell proliferation dynamically alters the secretion profile of cancer cells in the TME, which may play an important role in metastasis. As cancer cells proliferate, local cell density increases, which leads to an increase in tumor size that results in higher stage of the disease. Through local cell density, proliferation enhances cell migration in metastatic cells embedded in 3D matrices by increasing IL-6 and IL-8 levels. These observations also suggest that cell-density-dependent migration is unique to tumorigenic, metastatic cells exposed to 3D microenvironments that reconstitute features of tissues that enable in vitro recapitulation of in vivo function including spatiotemporal gradients of biochemical cues such as cytokines, chemokines and growth factors.

### Diagnostic, Prognostic, and Therapeutic Applications

Remarkably, our results demonstrate that IL-6 and IL-8 need to be present in the stoichiometric ratio of 5:2 to induce the enhanced the 3D cell migration. This pathway is able to significantly increase the migration of cells by 40% between low cell density and high cell density conditions. This change in velocity is significantly greater than those observed in previous 3D migration studies through the depletion of proteins. The results of this study also demonstrate that WASF3 is an important intermediate in this synergistic mechanism that induces cell-density-dependent migration. Earlier studies have shown that WASF3 is upregulated in the presence of IL-6 but not in the presence of IL-8. Strikingly, our results show that WASF3 expression is greatly upregulated in matrix embedded cells exposed to both IL-6 and IL-8. Clinical data also demonstrates that WASF3 and ARP2/3 are expressed in low levels in patients who have a longer survival rate (FIG. 5 E, F).

Our findings also surprisingly demonstrate that the levels of other secreted proteins that we assayed such as vascular endothelial growth factor (VEGF) and hepatocyte growth factor (HGF), which are both known to play important roles in tumor progression and tumor metastasis, did not increase with increasing cell density. These results suggest that metastatic cancer cells are able to independently produce IL-6 and IL-8 and do not require other cells in the TME such as fibroblastic stromal cells to supply them with the secreted proteins required to metastasize. These results also suggest the presence of a feedback signaling mechanism that is able to regulate many secreted molecules keeping them in an equilibrium. However the production of IL-6 and IL-8 is not regulated by this feedback signaling mechanism and continues to be produced as cell density increases.

The results further emphasize the necessity of 3D cultures in pharmaceutical studies as monolayer cell culture methods remain the de facto platform used in this field. Cells cultured in this flat platform often adopt physiologically irrelevant morphology and signaling patterns. For instance, the cell-density-dependent migration seen in matrix embedded cells is not observed in cells placed on 2D substrates. Our results suggest that Tocilizumab, a recombinant humanized, anti-human IL-6 receptor monoclonal antibody currently used in the treatment of Rheumatoid Arthritis , and Reparixin, an inhibitor of the IL-8 receptor, can together decrease the metastatic capacity of tumors by blocking the cognate receptors of IL-6 and IL-8, blocking the synergistic pathway responsible for enhanced cell migration and thereby inhibiting cancer cell migration.

Reparixin-Pub Chem CID: 9838712-IUPAC Name: (2R)-2-[4-(2-methylpropyl)phenyl]-N-methylsulfonylpropanamide or a compound, salt, solvate, or stereoisomer of any one of the compounds of Formula. I:

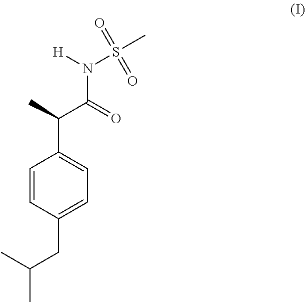 [SMILES: CC(C)Cc1ccc([C@@H](C)C(=O)NS(C)(=O)=O)cc1]

The results demonstrate that these two agents in a specific ratio are more effective than the agents alone. When the two agents are used alone a higher dosage is required to decrease cell migration. However, when the two agents are used in combination, lower dosages of the both agents are required. This result suggests that the combination of these two agents could be administered to cancer patients to prevent metastases from developing and thus improving patient outcomes.

Clinical data has demonstrated that both IL-6 and IL-8 are found at high concentrations in serum of patients with liver metastases and lung metastases, and that the serum concentrations of these two cytokines strongly correlate with the stage of cancer'. This information coupled with the results of this study indicate that the serum levels of IL-6 and IL-8 in in cancer patients can serve as a diagnostic and prognostic tool.

Embodiments of the disclosure concern methods and/or compositions for treating and/or preventing cancer in whereby modulation of the migration pathway is directly or indirectly related. In certain embodiments, individuals with a cancer such as lung or liver cancer are treated with a modulator (specifically an inhibitor) of the cancer cell migration pathway, and in specific embodiments an individual with cancer is provided an inhibitor of IL-6, IL-8, JAK2, STAT3, WASF3 or ARP2/3 Complex. An inhibitor of the present invention may decrease gene expression or inactive (or lower the activity of) the proteins produced from these genes.

In certain embodiments, an inhibitor of cancer cell migration may be any level so long as it provides amelioration of at least one symptom of a cancer, including lung or liver cancer. The level of cancer cell migration may decrease by at least 2, 3, 4, 5, 10, 25, 50, 100, 1000, or more fold expression compared to the level of cancer cell migration in a standard, or reference, in at least some cases.

An individual known to have cancer, suspected of having cancer, or at risk for having cancer may be provided an effective amount of an inhibitor of the present invention, including a combination of an IL-6 and an IL-8 inhibitor. Those at risk for cancer may be those individuals having one or more genetic factors, may be of advancing age, and/or may have a family history, for example.

In particular embodiments of the disclosure, an individual is given an agent for cancer therapy in addition to the one or more inhibitors of cancer cell migration, such as a combination of an IL-6 and an IL-8 inhibitor. Such additional therapy may include L-DOPA or dopamine receptor agonists and/or deep brain stimulation, for example. When combination therapy is employed with one or more inhibitors of cancer cell migration the additional therapy may be given prior to, at the same time as, and/or subsequent to the one or more inhibitors of cancer cell migration.

Pharmaceutical Preparations

Pharmaceutical compositions of the present invention comprise an effective amount of one or more inhibitors of cancer cell migration such as a combination of IL-6 inhibitor and IL-8 inhibitor, dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that comprises at least one inhibitor of cancer cell migration or additional active ingredient will be known to those of skill in the art in light of the present disclosure, as exemplified by Remington: The Science and Practice of Pharmacy, 21^{st} Ed. Lippincott Williams and Wilkins, 2005, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The inhibitors of cancer cell migration may comprise different types of carriers depending on whether it is to be administered in solid, liquid or aerosol form, and whether it need to be sterile for such routes of administration as injection. The present compositions can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The inhibitor of cancer cell migration may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present disclosure, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle compositions that include an inhibitor of cancer cell migration, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the inhibitor of cancer cell migration may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

A. Alimentary Compositions and Formulations

In one embodiment of the present disclosure, the inhibitors of cancer cell migration are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present disclosure may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

B. Parenteral Compositions and Formulations

In further embodiments, inhibitors of cancer cell migration may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by FDA Office of Biologics standards.

Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

C. Miscellaneous Pharmaceutical Compositions and Formulations

In other preferred embodiments of the invention, the active compound inhibitor of cancer cell migration may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725, 871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

Kits of the Disclosure

Any of the compositions described herein may be comprised in a kit. In a non-limiting example, an inhibitor of cancer cell migration (for example, a combination of IL-6 inhibitor and IL-8 inhibitor) may be comprised in a kit.

The kits may comprise a suitably aliquoted inducer of expression of PGC-1␣ and, in some cases, one or more additional agents. The component(s) of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the inhibitor of cancer cell migration and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. The inhibitor of cancer cell migration composition(s) may be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES/METHODS

### Cell Culture

Human fibrosarcoma HT1080 cells (ATCC) were cultured in Dulbecco's modified Eagle's medium (DMEM, Mediatech) supplemented with 10% (v/v) fetal bovine serum (FBS, Hyclone Laboratories), and 0.005% (w/v) gentamicin (Quality Biological). Human breast carcinoma MDA-MB-231 cells (ATCC) and MCF-7 cells (ATCC) were cultured in DMEM (Mediatech) supplemented with 10% FBS (Hyclone). Human glioblastoma U-87 MG cells (ATCC) were cultured in DMEM (Mediatech) supplemented with 10% FBS (Hyclone). Human diploid cell line, WI-38, (ATCC) were cultured in Eagle's minimal essential medium (EMEM, Mediatech) supplemented with 10% FBS (Hyclone). Human breast epithelial MCF10A cells (ATCC) and MCF 12A cells (ATCC) were cultured in DMEM supplemented with 5% horse serum (Atlanta biologicals), 20 ng/ml Human epidermal growth factor (Sigma-Aldrich),100 ng/ml cholera toxin, (Sigma-Aldrich) 0.01 mg/ml bovine insulin (Life technologies), and 500 ng/ml hydrocortisone

(Sigma-Aldrich). HT1080 cells transfected with shRNAs (see below) were grown in medium containing 1μg/ml puromycin. The cells were maintained at 37° C. and 5% CO₂ in a humidified incubator during cell culture and during live-cell microscopy.

Depletion of proteins with shRNAs

HT1080 cells were transfected as previously described in Giri et al. shRNA constructs targeting the Interleukin 6 and Interleukin 8 genes were purchased from Sigma Aldrich. After lentiviral-mediated transduction, Enzyme Linked Immunosorbent Assays (ELISA) were performed and only shRNAs showing more than 85% knockdown were used for subsequent studies (Supplementary information S1 K,L). They include:

SEQ ID NO: 1
(IL-6 sh59205)
ATCTCATTCTGCGCAGCTTTCTCGAGAAAGCTGCGCAGAATGAGAT
SEQ ID NO: 2
(IL-8 sh232053)
TGCGCCAACACAGAAATTATTCTCGAGAATAATTTCTGTGTTGGCGCA;
SEQ ID NO: 3
(IL-6R sh289773)
CCGGCCAGTCCAGATATTTCACATTCTCGAGAATGTGAAATATCTGGA
CTGG;
SEQ ID NO: 4
(IL-8R sh37836)
CCGGGAAGCGCTACTTGGTCAAATTCTCGAGAATTTGACCAAGTAGCG
CTTCTT;

The transfected cells were embedded in type I collagen matrices and incubated overnight at 37° C. and 5% CO2 in a humidified incubator. The conditioned media from the cells were collected and filtered through a 0.45 μm filter (Millipore) to remove cell debris. The total quantity of IL-6 and IL-8 produced by the cells were measured using Human quantikine ELISA kits (R&D system.

### 3D Collagen Matrix

HT1080 cells were embedded in 2mg/ml type I collagen gel as described previously by Fraley et al²⁰. Briefly, cell suspensions containing 5000 to 75,000 cells in 1:1 (v/v) ratio of cell culture media and reconstitution buffer were mixed with appropriate volume of soluble rat-tail collagen I (Corning Inc.) to obtain a final collagen I concentration of 2 mg/ml. A calculated amount of 1M NaOH was added quickly and the final solution was mixed well to bring the pH to ˜7. The cell suspensions was added to a 24-well coverslip-bottom cell-culture dish and immediately transferred to an incubator maintained at 36° C. to allow polymerization. Fresh medium was added 1 h before imaging. MDA MB 231 and U-87 cells were embedded in 1 mg/ml type I collagen matrix

### Velocity and Protrusion Topology of Matrix Embedded Cells

Phase-contrast images of matrix-embedded cells were recorded 2 min apart for 16.5 h using a Cascade 1K CCD camera (Roper Scientific) mounted on a Nikon TE2000 microscope with a 10× objective lens. Single cells were tracked using Metamorph imaging software. A custom MATLAB program calculated the velocity for each cell using the x- and y-coordinates obtained from tracking data using the following equation:

$$Speed = \frac{\sqrt{〈 ⌈ x ( t + Δ t ) - x ( t ) ⌉ 2 + ⌈ y ( t + Δ t ) - y ( t ) ⌉ 2 〉}}{t} .$$

For the characterization of protrusion topology, the movies were used to count the total number of mother protrusions, and the number of first-, second-, and third-generation protrusions generated by the cell (e.g. FIG. 1, C). The protrusions emanating directly from the cell body, even when split, were termed mother protrusions; protrusions originating from the mother protrusions were termed first-generation, and so on. Mitotic cells were not included in the measurements.

Proliferation assays

HT1080 cells were embedded in type I 3D collagen matrices in increasing cell numbers from 5000 to 60,000 cells. Phase contrast images of the cells were recorded 8 min apart for 48 h. The average doubling time was obtained by measuring the time between the 1^{st} and 2^{nd} divisions. Cell viability assay using Prestoblue (Invitrogen) was also conducted on the matrix-embedded cells of increasing cell number. Fluorescence was measured every 6 h for 48 h.

Collagen inter-fiber Spacing and Alignment

Matrix embedded cells with cell densities ranging from 10 cells/mm³ to 150 cells/mm³ were imaged and analyzed according to the methods highlighted in Fraley et al to determine the inter-fiber spacing and alignment.

### Condition Medium and High Throughput Secretomic Analysis

Matrix-embedded cells with cell densities ranging from 10 cells/mm³ to 150 cells/mm³ were incubated for 24 h at 37° C. in a humidified incubator. The conditioned medium from the cells was then collected and filtered through a 0.45 μm filter (Millipore) to remove cell debris. High throughput secretomic analysis was conducted on the condition medium collected as described previously by Lu et al.

Conditioned medium from HT1080 cells embedded in matrices with a cell density of 50 cells/mm³ was added to freshly made matrices with a cell density of 10 cells/mm³. These conditions were replicated when extracting conditioned medium from the HT1080 transfected cells.

### Biochemical Agents

Recombinant IL-6 and IL-8 (R&D systems) reconstituted in DPBS (Life technologies) were added to matrix embedded cells with a cell density of 10 cells/mm³ and imaged as described above (See velocity and protrusion topology of matrix embedded cells). Matrix embedded cells with cell densities of 10 cells/mm³ and 50 cells/mm³ were exposed to specific IL-6 and IL-8 functional antibodies (Proteintech) at a concentration of 0.5 μg/mL. Matrix embedded cells with cell densities of 10 cells/mm³ and 50 cells/mm³ were exposed to Tocilizumab (Roche) and Reparixin (Dompe pharmaceuticals) STAT3 and WASF3 activity

Matrix embedded cells with cell densities of 10 cells/mm³ and 50 cells/mm³ were incubated for 24h at 37° C. in a humidified incubator. The matrices were exposed to cell lysis buffer and mechanically broken down using a syringe. The suspension was centrifuged and the supernatant was measured for STAT3 and PhosphoSTAT3 using an ELISA kit (Abcam). WASF3 expression was measured using qRT-PCR for the cell densities stated previously and matrix embedded cells with a cell density of 10 cells/mm³ exposed to IL-6 and IL-8 alone and in combination at the precise concentrations found at a high cell density of 50 cells/mm³. Total RNA isolation was performed with RNA MiniPrep kit (Zymo research). cDNA synthesis was carried out as previously described by Gilkes et al⁴⁹. The sequence for the cDNA primers that were used during PCR are found in the table below.

| SEQ ID NO: 5 | |
|---|---|
| (HS-18S-FOR) | gaggatgaggtggaacgtgt |
| SEQ ID NO: 6 | |
| (HS-18S-REV) | agaagtgacgcagccctcta |
| SEQ ID NO: 7 | |
| (HS-WASF3-FOR) | aagggattaccagcgaacttg |
| SEQ ID NO: 8 | |
| (HS-WASF3-REV) | cttcagcatgtttgctcagact |
| SEQ ID NO: 9 | |
| (HS-ARP2/3-FOR) | aacgacacaacaagccggaa |
| SEQ ID NO: 10 | |
| (HS-ARP2/3-REV) | tggagccctcaatcagaacct |

Inhibitor Assays

Matrix embedded with cell densities of 10 cells/mm³ and 50 cells/mm³ were exposed to JAK2 inhibitor, AG-490 (Santa Cruz Biotechnology), STAT3 inhibitor, S31-201, (Santa Cruz Biotechnology) and ARP 2/3 complex inhibitor, CK 666, (Sigma-Aldrich) for 1 h before cells were imaged as described above. (See velocity and protrusion topology of matrix embedded cells)

### Statistics

The mean values ±SE were calculated and plotted using GraphPad Prism software (GraphPad Software). One-way ANOVA test was performed to determine statistical significance, which is indicated in the graphs using a Michelin grade scale ***p<0.001, ***p<0.01, and *p<0.05.

1. A method of inhibiting cancer cell migration comprising the following steps:
a. providing a mixture comprising an IL-6 inhibitor and an IL-8 inhibitor;
b. applying the mixture to cancer cells; and
c. inhibiting the migration of the cancer cells.

2. The method of claim 1, wherein the method is performed in vitro.

3. The method of claim 1, wherein the method is performed in vivo.

4. The method of claim 1, wherein the IL-6 inhibitor inhibits IL-6 gene expression, IL-6 protein activity, or both.

5. The method of claim 1, wherein the IL-6 inhibitor is a shRNA.

6. The method of claim 5, wherein the shRNA is selected from the group consisting of SEQ ID NO: 1, SEQ ID NO: 3, or a combination thereof.

7. The method of claim 1 wherein the IL-6 inhibitor is an antibody.

8. The method of claim 1 wherein the IL-6 inhibitor is a recombinant humanized, anti-human IL-6 receptor monoclonal antibody.

9. The method of claim 1, wherein the IL-8 inhibitor inhibits IL-8 gene expression, IL-6 protein activity, or both.

10. The method of claim 1, wherein the IL-8 inhibitor is a shRNA.

11. The method of claim 10, wherein the shRNA is selected from the group consisting of SEQ ID NO:2, SEQ ID NO:4, or a combination thereof.

12. The method of claim 1, wherein the IL-8 inhibitor is an antibody.

13. The method of claim 1, wherein the IL-8 inhibitor is an inhibitor of the IL-8 receptor.

14. The method of claim 13 wherein the IL-8 inhibitor is a (2R)-2-[4-(2-methylpropl)phenyl]-N-methylsulfonylpropanamide.

15. The method of claim 1 wherein the cancer cells are liver metastases cells.

16. The method of claim 1 wherein the cancer cells are lung metastases cells.

17. The method of claim 1 wherein the inhibiting the migration of the cancer cells is observed when compared to reference cancer cells that are substantially free of the IL-6 inhibitor and the 1L-8 inhibitor.

18. A method of determining cancer patient longevity comprising:
a. providing a biological sample from a cancer patient;
b. placing the biological sample in contact with an IL-6 and IL-8 binding agent; and
c. determining the concentration of IL-6 and IL-8 in the biological sample.

19. The method of claim 18, wherein cancer patient longevity is enhanced when the concentration ratio of IL-6/IL-8 in the biological sample is similar to that seen in patients without cancer.

20. The method of claim 18, wherein the binding agent is an antibody.

21. A method or treating or preventing cancer in a subject comprising the following steps:
a. providing a mixture comprising an IL-6 and an IL-8 inhibitor; and
b. administering the mixture to a subject with cancer; and
c. treating or preventing cancer in the subject.

22. The method of claim 21, wherein the IL-6 inhibitor inhibits IL-6 gene expression, IL-6 protein activity, or both.

23. The method of claim 21, wherein the IL-6 inhibitor is a shRNA.

24. The method of claim 23, wherein the shRNA is selected from the group consisting of SEQ ID NO: 1, SEQ ID NO: 3, or a combination thereof.

25. The method of claim 21 wherein the IL-6 inhibitor is an antibody.

26. The method of claim 21 wherein the IL-6 inhibitor is a recombinant humanized, anti-human IL-6 receptor monoclonal antibody.

27. The method of claim 21, wherein the IL-8 inhibitor inhibits IL-8 gene expression, IL-6 protein activity, or both.

28. The method of claim 21, wherein the IL-8 inhibitor is a shRNA.

29. The method of claim 28, wherein the shRNA is selected from the group consisting of SEQ ID NO:2, SEQ ID NO:4, or a combination thereof.

30. The method of claim 21, wherein the IL-8 inhibitor is an antibody.

31. The method of claim 21, wherein the IL-8 inhibitor is an inhibitor of the IL-8 receptor.

32. The method of claim 31 wherein the IL-8 inhibitor is a (2R)-[4-(2-methylpropyl)phenyl]-N-methylsulfonylpropanamide.

33. The method of claim 21 wherein the cancer is a liver metastases.

34. The method of claim 21 wherein the cancer is a lung metastases.

35. A method for inhibiting cancer cell migration comprising the following steps:
a. providing a mixture comprising an inhibitor of a gene selected from the group consisting of JAK2, STAT3, WASF3, ARP2/3 complex or a combination thereof;
b. applying the mixture to cancer cells; and
c. inhibiting the migration of the cancer cells.

36. The method of claim 35 wherein the inhibiting of the cancer cells is observed when compared to reference cancer cells that are substantially free of the inhibitor.
